(19) United States
(12) Patent Application Publication
(10) Pub. No.: US 2019/0290214 A1
(43) Pub. Date: Sep. 26, 2019

(54) Health Management Mobile Terminal, Method, and Server

(71) Applicant: YANHUANG DONGFANG (BEIJING) HEALTHCARE TECHNOLOGY CO.,LTD., Beijing (CN)

(72) Inventor: Zhonghua CI, Beijing (CN)

(21) Appl. No.: 15/954,279

(22) Filed: Apr. 16, 2018

(30) Foreign Application Priority Data
Mar. 22, 2018 (CN) 2018102419770

(51) Int. Cl. A61B 5/00; G10L 25/54; A61B 5/0205; A61B 5/1455; A61B 5/145; G16H 40/67

(52) U.S. Cl. CPC A61B 5/742; G10L 25/54; A61B 5/0022; A61B 5/0205; A61B 5/14551; H04W 4/80; A61B 5/14532; A61B 5/4803; A61B 5/4854; G16H 40/67; A61B 5/02055

(57) ABSTRACT

The present disclosure discloses a health management mobile terminal, method, and server. The mobile terminal includes a display module, a microprocessor module, a communication module, and a medical module; the medical module is used to collect and monitor health information of a user; the microprocessor module is used to acquire health data of the user; the communication module is used to send the health data to a server in communication connection therewith, and receive a health analysis result acquired after the server analyzes the health data; the display module is used to perform detecting item selection, and display the health data and the health analysis result. In the present disclosure, the user can detect his/her own physical health situation at any time; in combination with the usage of smart cellphone, corresponding detection numerical values and graphs can be intuitively displayed through the display module.

### Technical Field

The present disclosure relates to the field of wise medical treatment, and particularly to a health management mobile terminal, method, and Server.

### BACKGROUND

With further development of economy and society, people's living pace and pressure is further increased. The latest medical research report shows that the morbidity of heart diseases is on the rise, and functions of lungs and blood are also decreased. Dysfunction of gas ventilation and transfer of lungs and a low content of hemoglobin both will lead to reduction of oxygen content in the blood.

In the above, the heart diseases have the characteristics of being difficult to diagnose in early stage, sudden attack, and high fatality rate. They have become number one enemy threatening people's health in our country. In order to timely prevent occurrence of the heart diseases, an electrocardio waveform should be monitored in real time. When the oxygen content in the blood is lower than 95%, syndromes such as polypnea, fatigueness, or headache will appear, and other diseases will be caused.

Thus, a portable electrocardio and blood oxygen monitoring apparatus is needed to be capable of monitoring relevant parameters in real time without affecting people's normal life.

Besides, the inventor believes that the existing apparatus merely can monitor corresponding data, but cannot give specific health situation, diagnosis of illness, and other recommendations helping the user improve the health condition.

### SUMMARY

An object of the present disclosure is to provide a health management mobile terminal, method, and server with regard to the problems existing in the related art.

In order to achieve the above object, the present disclosure provides a health management mobile terminal, including a display module, a microprocessor module, a communication module, and a medical module;

the medical module is used to collect and monitor health information of a user;

the microprocessor module is used to process the health information, and acquire health data of the user;

the communication module is used to send the health data to a server in communication connection therewith, and receive a health analysis result acquired after the server analyzes the health data;

the display module is used to perform detecting item selection, and display the health data and the health analysis result.

Furthermore, with respect to the health management mobile terminal as described above, the medical module includes a blood oxygen detecting sub-module and an electrocardio detecting sub-module;

the electrocardio detecting function sub-module is used to detect and acquire electrocardio data and heart rate data of the user;

the blood oxygen detecting sub-module is used to detect and acquire blood oxygen data of the user;

the health data includes: a heart rate value, an electrocardiogram, and a blood oxygen value.

Furthermore, with respect to the health management mobile terminal as described above, the health management mobile terminal further includes a man-machine interaction interface;

the man-machine interaction interface is used to perform the detecting item selection, and further used to perform user management; the user management includes control over data collection, data processing, data transmission, and display.

Furthermore, the health management mobile terminal as described above further includes a Bluetooth module and a memory module; the Bluetooth module is electrically connected to the microprocessor module, for externally connecting a blood pressure detector, a breath detector, a body temperature detector, and a glucose detector, and performing data transmission; the memory module is electrically connected to the microprocessor module, and is used to store the health analysis result.

Furthermore, with respect to the health management mobile terminal as described above, the health management mobile terminal further includes: a voice collecting module and a voice processing module;

the voice collecting module is used to collect voice information of the user, and acquire a basic frequency of the user voice;

the voice processing module is used to determine a voice basic frequency interval of the user according to the basic frequency of the voice, and acquire music matched with the voice basic frequency interval.

Furthermore, with respect to the health management mobile terminal as described above, the health management mobile terminal further includes a music matching module;

the music matching module inquires music corresponding to the voice basic frequency interval from a preset music database and pushes the same.

The present disclosure further provides a health management method, including:

performing detecting item selection;

collecting and monitoring health information of a user;

processing the health information, and acquiring health data of the user;

sending the health data to a server in communication connection therewith, and receiving a health analysis result acquired after the server analyzes the health data;

displaying the health data and the health analysis result.

Furthermore, the health management method as described above further includes:

collecting voice information of the user, and acquiring the basic frequency of a user voice;

determining a voice basic frequency interval of the user according to the basic frequency of the voice, and acquiring music matched with the voice basic frequency interval;

inquiring music corresponding to the voice basic frequency interval from a preset music database and pushing the same.

The present disclosure further provides a health management server, wherein the server is configured to:

receive health data sent by a mobile terminal, and send to the mobile terminal a health analysis result acquired after the health data is analyzed.

Furthermore, with respect to the health management server as described above, the server includes an analyzing module and a storage module;

the analyzing module is used to acquire the health analysis result after analyzing the health data;

the storage module is used to store the health data and the health analysis result.

The beneficial effects of the present disclosure are as follows:

in the present disclosure, the functions of electrocardio detection and blood oxygen detection are simultaneously integrated in a cellphone, so that the user can detect his/her own physical health situation at any time; in combination with the usage of smart cellphone, corresponding detection numerical values and graphs can be intuitively displayed through the display module.

On one hand, the present disclosure has the characteristic of convenience, being capable of performing the detection at set intervals, and accumulating more detection numerical values to provide comparatively perfect data support to overall health evaluation; on the other hand, the detection results can be analyzed by the server, to further acquire the health condition of the user, moreover, the health management also can be carried out for the user according to the health condition, and by matching corresponding health products and insurance products, the health products in various aspects such as food and drink, medical equipment, and illness insurance can be recommended to the user; therefore, the present disclosure has the functions of finding health problems timely,providing a targeted health management and providing solutions.

### BRIEF DESCRIPTION OF DRAWINGS

In order to illustrate the technical solutions of embodiments of the present application or the related art more clearly, accompanying drawings which need to be used in the description of the embodiments will be briefly described below. Apparently, the accompanying drawings described in the following are merely for some embodiments of the present disclosure, and a person ordinarily skilled in the art still can obtain other accompanying drawings according to these accompanying drawings.

FIG. 1 is a structural diagram of a health management mobile terminal shown according to an embodiment;

FIG. 2 is a flow chart of a health management method;

FIG. 3 is a schematic diagram of a modular structure of a server in a health management mobile terminal shown according to an embodiment.

### DETAILED DESCRIPTION OF EMBODIMENTS

In order to enable a person skilled in the art to better understand the solutions of the present application, the technical solutions of the embodiments of the present application will be described clearly and completely below with reference to the accompanying drawings of the embodiments of the present application. Apparently, the embodiments described are merely for some of the embodiments of the present application, rather than all of the embodiments. All the other embodiments that are obtained by a person skilled in the art without inventive effort on the basis of the embodiments of the present application shall be covered by the protection scope of the present application.

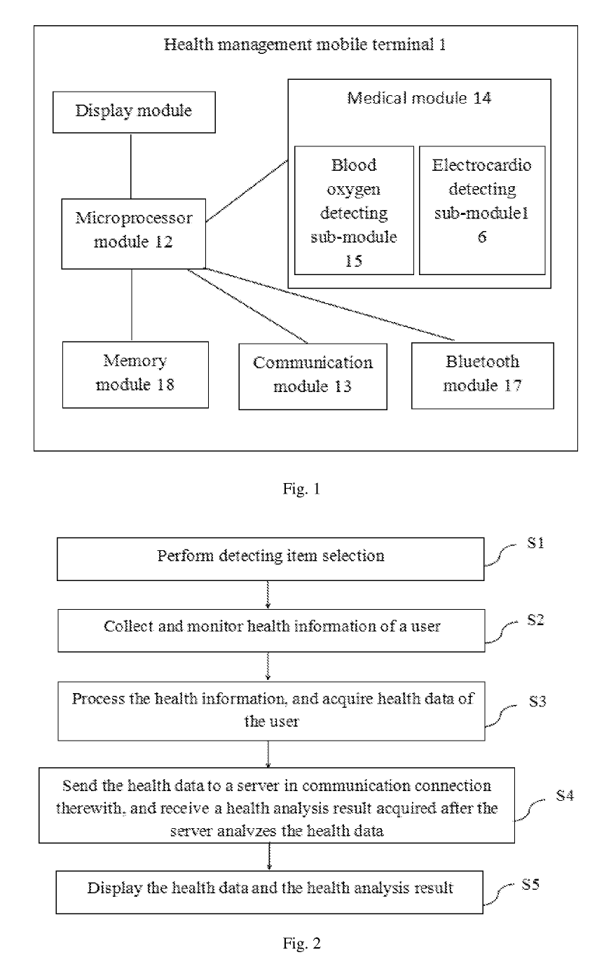

As shown in FIG. 1, the present disclosure provides a health management mobile terminal, including a display module 11, a microprocessor module 12, a communication module 13, and a medical module 14;

the medical module 14 is used to collect and monitor health information of a user;

the microprocessor module 12 is used to process the health information, and acquire health data of the user;

the communication module 13 is used to send the health data to a server in communication connection therewith, and receive a health analysis result acquired after the server analyzes the health data;

the display module 11 is used to perform detecting item selection, and display the health data and the health analysis result.

In some embodiments, the medical module includes a blood oxygen detecting sub-module 15 and an electrocardio detecting sub-module 16;

the blood oxygen detecting sub-module 15 is used to detect and acquire blood oxygen data of the user;

the electrocardio detecting sub-module 16 is used to detect and acquire electrocardio data and heart rate data of the user;

the health data includes: a heart rate value, an electrocardiogram, and a blood oxygen value.

In some embodiments, with respect to the health management mobile terminal as described above, the health management mobile terminal further includes: a man-machine interaction interface;

the man-machine interaction interface is used to perform the detecting item selection, and further used to perform user management; the user management includes control over data collection, data processing, data transmission, and display.

Registration information registered by the user, after being input through the man-machine interaction interface, is sent to the server through a first communication module to be stored; account login information, after being input, is sent to the server through the first communication module to be verified, and after the verification is passed, the man-machine interaction interface enters a detecting item selection interface, and performs operation display of local information.

In some embodiments, the health management mobile terminal as described above further includes a Bluetooth module 17. The Bluetooth module 17 is electrically connected to the microprocessor module 12, for externally connecting a blood pressure detector, a breath detector, a body temperature detector, and a glucose detector, and performing data transmission.

In some embodiments, the health management mobile terminal as described above further includes a memory module 18. The memory module is electrically connected to the microprocessor module, and is used to store the health analysis result.

In some embodiments, the health management mobile terminal further includes: a voice collecting module and a voice processing module;

the voice collecting module is used to collect voice information of the user, and acquire a basic frequency of the user voice;

the voice processing module is used to determine a voice basic frequency interval of the user according to the basic frequency of the voice, and acquire music matched with the voice basic frequency interval; specifically, after the present embodiment is combined with the traditional Chinese medicine, a five-note type of the user can be determined according to the voice basic frequency interval, wherein the five-note type is one from gong, shang, jue, zhi, and yu.

Specifically, different users have different five-note types, meanwhile, different five-note types are corresponding to different physiological symptoms. Therefore, the physiological symptoms of the current wearer can be determined according to the five-note type of the current user; since different five-note types have been matched with the music in advance, the music matched with this five-note type can be acquired at this time; the music is played.

In an embodiment of the present disclosure, a technician can set a basic frequency range corresponding to each five-note type in a terminal or the server 2 in advance, and each five-note type is combined with its corresponding basic frequency range into one record to be stored in a first corresponding relationship between the five-note type stored in the terminal or the server 2 and the basic frequency range interval. A first column in the first corresponding relationship stores the five-note type, and a second column stores the basic frequency range interval.

In some embodiments, the health management mobile terminal further includes a music matching module;

the music matching module inquires music corresponding to the voice basic frequency interval from a preset music database and pushes the same.

As shown in FIG. 2, the present disclosure further provides a health management method, including:

S1: performing detecting item selection;

S2: collecting and monitoring health information of a user;

S3: processing the health information, and acquiring health data of the user;

S4: sending the health data to a server in communication connection therewith, and receiving a health analysis result acquired after the server analyzes the health data;

S5: displaying the health data and the health analysis result.

In some embodiments, the health management method as described above further includes:

collecting voice information of the user, and acquiring the basic frequency of a user voice;

determining a voice basic frequency interval of the user according to the basic frequency of the voice, and acquiring music matched with the voice basic frequency interval;

inquiring music corresponding to the voice basic frequency interval from a preset music database and pushing the same.

The health management mobile terminal 1 in the present embodiment can send or receive a signal through a wired or wireless network or in other manners. A person skilled in the art can easily expand that the health management mobile terminal 1 still can be expanded to a tablet computer, a portable E-mail device, an E-book, a hand-held game machine and/or a game controller, a notebook computer, a netbook, a hand-held electronic device, a smart wearable device, and so on. The present disclosure covers any suitable mobile communication device. The mobile communication device can enable a user using the present mobile communication device to access network.

The present disclosure further provides a health management server. The server 2 is used to receive health data sent by a mobile terminal, and send to the mobile terminal a health analysis result acquired after the health data is analyzed.

In some embodiments, the server includes an analyzing module and a storage module;

the analyzing module 21 is used to acquire the health analysis result after analyzing the health data;

the storage module 22 is used to store the health data and the health analysis result.

The server stores heart rate data, electrocardiogram, and blood oxygen data measured by the user each time, because the numerical value of one time is more likely to be unique and cannot accurately reflect the health condition of the user, therefore, it is statistically more accurate to acquire the physical condition of the user through analysis of a lot of numerical values.

The server 2 in the present disclosure should be construed as a point of service providing processing, database, and communication facility. For example, the server 2 can refer to a single physical processor having relevant communication and data storage and database facility, or it can refer to an aggregation of networking or clustered processors, relevant networks, and storage devise, and operate software and one or more database systems, and application software of supporting services provided by the server 2. The server 2 may differ considerably in configuration or performance, but the server 2 generally can include one or more central processing units and a memory. The server 2 further includes one or more mass storage devices, one or more power supplies, one or more wired or wireless network interfaces, one or more input/output interfaces, or one or more operation systems, such as Windows Server, Mac OS X, Unix, Linux, and FreeBSD, and so on.

According to some embodiments of the present disclosure, the server 2 can be an integrated server 2 or a decentralized server 2 across multiple computers or computer data centers. The server 2 can be of various types, for example, but not limited to, network server 2, news server 2, mail server 2, message server 2, advertisement server 2, file server 2, application server 2, interaction server 2, database server 2, or proxy server 2. In some embodiments, each server 2 can include hardware, software or embedded logic component for implementing suitable functions supported or realized by the server 2 or combination of two or more such components. In the present disclosure, the server 2 is used to provide all the functions necessary for a service terminal.

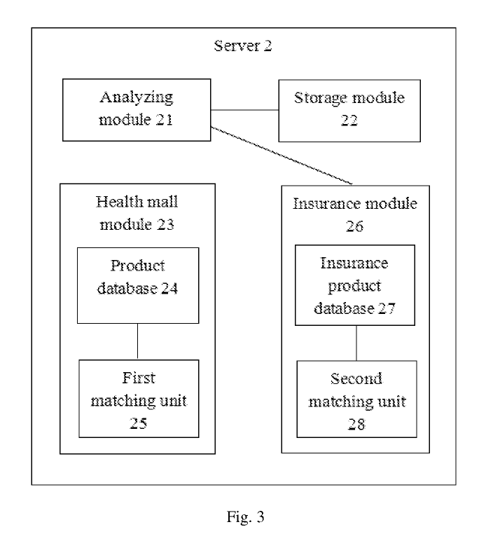

As shown in FIG. 3, in some embodiments, the server 2 further includes a health mall module 23; the health mall module 23 is used to select a health product matched with the health condition of the user according to the health condition of the user. In the present embodiment, the health product can include, but is not limited to, beverage, food, health care product, medical equipment, and so on.

The health mall module 23 includes a product database 24 and a first matching unit 25;

the product database 24 is used to store product names, images, and efficacy information;

the first matching unit 25 is used to match the health condition with data in the product database to obtain a matched health product.

The health condition analyzed and obtained by the server in the present embodiment can be reflected by specific characteristic words, for example, low immunity and sub-health; the products in the product database also have characteristic words corresponding to the health condition as product information; the server 2 gives a corresponding matched product by matching the characteristic words of the health condition with the product information in the product database, and pushes the same to the man-machine interaction interface of the health management mobile terminal of the user; for example, low immunity is taken as the product information of dendrobium officinale, therefore, the user with low immunity will receive the product recommendation of dendrobium officinale. Therefore, the recommendation can be made targetedly, so that the user obtains a better health management function.

As shown in FIG. 3, in some embodiments, the server 2 further includes an insurance module 26:

the insurance module 26 is used to select an insurance product matched with the health condition of the user according to the health condition of the user.

The insurance module 26 includes an insurance product database 27 and a second matching unit 28:

the insurance product database 27 is used to store an insurance product list;

the second matching unit 28 is used to match the health condition information with data in the insurance product database to obtain a matched insurance product.

The health condition analyzed and obtained by the server in the present embodiment can be reflected by specific characteristic words, for example, good health condition, ordinary health condition, and poor health condition; the products in the insurance product database also have characteristic words corresponding to the health condition as insurance product information, for example, poor health condition is taken as the insurance product information of critical illness insurance; the server 2 gives the corresponding matched insurance product by matching the characteristic words of the health condition with the insurance product information in the insurance product database, and recommends the same to the user; therefore, the recommendation also can be made targetedly, further ensuring the health of the user in a way of insurance.

The above merely describes some exemplary examples of the present invention in an illustrative manner. It goes without saying that a person ordinarily skilled in the art can modify the examples described through various different manners without departing from the spirit and scope of the present invention. Therefore, the above accompanying drawings and description are essentially illustrative, and should not be construed as limitation to the scope protected by the claims of the present invention.

What is claimed is:

1. A health management mobile terminal, comprising a display module, a microprocessor module, a communication module, and a medical module,
wherein the medical module is used to collect and monitor health information of a user;
the microprocessor module is used to process the health information, and acquire health data of the user;
the communication module is used to send the health data to a server in communication connection therewith, and receive a health analysis result acquired after the server analyzes the health data;
the display module is used to perform detecting item selection, and display the health data and the health analysis result.

2. The health management mobile terminal of claim 1, wherein the medical module includes a blood oxygen detecting sub-module and an electrocardio detecting sub-module;
the blood oxygen detecting sub-module is used to detect and acquire blood oxygen data of the user;
the electrocardio detecting sub-module is used to detect and acquire electrocardio data and heart rate data of the user;
the health data includes: a heart rate value, an electrocardiogram, and a blood oxygen value.

3. The health management mobile terminal of claim 1, wherein the health management mobile terminal further comprises: a man-machine interaction interface;
the man-machine interaction interface is used to perform the detecting item selection, and further used to perform user management; the user management comprises control over data collection, data processing, data transmission, and display.

4. The health management mobile terminal of claim 1, further comprises a Bluetooth module and a memory module; the Bluetooth module is electrically connected to the microprocessor module, for externally connecting a blood pressure detector, a breath detector, a body temperature detector, and a glucose detector, and performing data transmission; the memory module is electrically connected to the microprocessor module, and is used to store the health analysis result.

5. The health management mobile terminal of claim 1, wherein the health management mobile terminal further comprises: a voice collecting module and a voice processing module;
the voice collecting module is used to collect voice information of the user, and acquire a basic frequency of the user voice;
the voice processing module is used to determine a voice basic frequency interval of the user according to the basic frequency of the voice, and acquire music matched with the voice basic frequency interval.

6. The health management mobile terminal of claim 1, wherein the health management mobile terminal further comprises a music matching module;
the music matching module inquires music corresponding to the voice basic frequency interval from a preset music database and pushes the same.

7. A health management method, comprising:
performing detecting item selection;
collecting and monitoring health information of a user;
processing the health information, and acquiring health data of the user;
sending the health data to a server in communication connection therewith, and receiving a health analysis result acquired after the server analyzes the health data;
displaying the health data and the health analysis result.

8. The health management method of claim 7, further comprising:
collecting voice information of the user, and acquiring the basic frequency of a user voice;
determining a voice basic frequency interval of the user according to the basic frequency of the voice, and acquiring music matched with the voice basic frequency interval;
inquiring music corresponding to the voice basic frequency interval from a preset music database and pushing the same.

9. A health management server, wherein the server is configured to:
receive health data sent by a mobile terminal, and send to the mobile terminal a health analysis result acquired after the health data is analyzed.

10. The health management server of claim 9, wherein the server comprises an analyzing module and a storage module;
the analyzing module is used to acquire the health analysis result after analyzing the health data;
the storage module is used to store the health data and the health analysis result.
